(19) United States
(12) Patent Application Publication
(10) Pub. No.: US 2005/0214832 A1
(43) Pub. Date: Sep. 29, 2005

(54) Method and kit for performing functional tests on biological cells

(76) Inventors: Hugo Haemmerle, Tuebingen (DE); Thomas Joos, Tuebingen (DE); Markus Templin, Tuebingen (DE); Hansjurgen Volkmer, Tuebingen (DE); Kerstin Ragnitz, Gomaringen (DE); Susanne Stumpf, Kusterdingen (DE); Cornelia Kuschel, Moessingen (DE)

Correspondence Address: MORRISON & FOERSTER LLP, 3811 VALLEY CENTRE DRIVE, SUITE 500, SAN DIEGO, CA, 92130-2332, US

(21) Appl. No.: 11/051,315

(22) Filed: Feb. 4, 2005

Related U.S. Application Data
(63) Continuation of application No. PCT/EP03/07440, filed on Jul. 9, 2003.

(30) Foreign Application Priority Data
Aug. 5, 2002 (DE) 102 36 101.0

(51) Int. Cl. C12Q 1/68; G01N 33/53; G01N 33/567

(52) U.S. Cl. 435/6; 435/7.2

(57) ABSTRACT

A process for carrying out functional assays on test cells is described comprising the following steps: a) providing an array of measuring points which are separate from each other and to which in each case capture molecules to which said test cells can bind are immobilized, b) generating a mixture of said test cells and of reference particles which are capable of binding to said capture molecules and which are distinguishable from said test cells, c) contacting the mixture of step b) with the array so that test cells and reference particles can bind to each measuring point, and d) determining the ratio of bound test cells of interest to bound reference particles for at least some of the measuring points. In a further process, at least one measuring point with its assigned capture molecules is distributed between a plurality of measuring areas in the array, which are arranged at various sites in said array. In another process, the array is agitated, after contacting with the test cells and, where appropriate, the reference particles.

### CROSS-REFERENCE TO RELATED APPLICATIONS

This application is a continuation of copending international patent application PCT/EP2003/007440 filed on Jul. 9, 2003 and designating the U.S., which was not published under PCT Article 21(2) in English, and claims priority of German patent application DE 102 36 101.0 filed on Aug. 5, 2002, both of which are incorporated herein by reference.

### FIELD OF THE INVENTION

The present invention relates to a process and to a kit for carrying out functional assays on biological cells, which process comprises using an array of measuring points, with at least one capture molecule or binding partner for the biological cells to be assayed being immobilized on each measuring point.

“Functional assay” means, within the scope of the present invention, by way of example but not by way of conclusion or limitation, those experiments, assays or measurements in which particular properties or features of the cells or the change in said properties or features are recorded or evaluated as a function of a treatment of said cells and/or the type of capture molecules and/or the addition of substances.

Treatment of the cells comprises, for example, irradiation with a high energy radiation as used, for example, in radiotherapy. The addition of substances comprises, for example in the context of pharmaceutical screening, the administration of pharmaceutical preparations whose effect on the cells is studied, for example, in a dose-finding study, or the addition of antibodies which are screened for cell surface receptors. The choice of the type of capture molecules relates, for example, to components of extracellular matrix molecules for simulating the natural microenvironment of the cells, in order to assay in vitro their reaction to radiation, stimulation by ligands and/or added pharmaceuticals under conditions of the natural microenvironment.

“Biological cells to be assayed” means within the scope of the present invention, by way of example but not by way of conclusion or limitation, primary animal, in particular human, cells, plant or bacterial cells, cell lines, genetically modified cells, cells from biopsy material, healthy nondegenerated cells, in particular tumor cells, peripheral blood cells, etc. These cells are referred to as test cells hereinbelow.

“Properties and features” of the test cells include by way of example but not by way of conclusion or limitation their ability to proliferate, their viability, the pattern of their cell surface molecules, their ability to exchange signals or to interact with other cells, a possible pathological condition, a genetic degeneration, their genetic profile, their expression profile, their ability to bind to particular substances.

### RELATED PRIOR ART

These processes are carried out by using arrays of measuring points in order to be able to read out different cellular functions in parallel, using a small number of cells. An advantage of such arrays is the fact that, in contrast to microtiter plates, the same environmental conditions prevail for all measuring points.

Carrier plates with arrays suitable for carrying out processes of this kind and examples of functional assays of this kind can be found, for example, in WO 02/02226 of the applicant or in WO 00/39580.

Said carrier plates are usually glass or plastic plates which have a functionalized, for example aldehyde-activated, surface on which capture molecules or binding partners for the test cells are immobilized at measuring points separated from one another. The surface is blocked between the measuring points in order to prevent unspecific binding of-test cells or other substances.

The measuring points are from 200 to 800 μm in diameter and the distance between their centers is, for example, 500 μm so that an area of 1×1 cm can hold 100 measuring points.

For example, different capture molecules are immobilized on the measuring points. A solution containing test cells is then applied to the carrier plate and then incubated for a particular time, before the test cells which have not immobilized to capture molecules are washed off again. The bound test cells are then recorded optically in a space-resolved manner in order to determine to which capture molecules said test cells have bound. Optical recording may be carried out, for example, via bright field microscopy or fluorescence measurements but it is also possible to use other principles of measurement as described, for example, in WO 02/02226 or WO 00/39580.

Measurements carried out by the inventors of the present application using carrier plates of this kind have now revealed that the measured signals between different measuring points within one carrier plate and between measuring points of different carrier plates vary greatly, although the capture molecules, the test cells and the experimental approach were identical. This variability between the measuring points on a carrier plate and between different carrier plates makes it frequently impossible to compare the results of the measurement with one another in a sufficiently reliable manner.

This problem can also be found in the publication by Belov et al.: “Immunophenotyping of Leukemias Using a Cluster of Differentiation Antibody Microarray”, in Cancer Research (2001) 61, 4483-4489.

This publication describes a process in which more than 50 CD antigens on leukocytes are detected. For this purpose, use is made of an array of various antibodies to the particular CD antigens, which are immobilized on different measuring points, to which array a suspension of test cells is applied, and said test cells bind only to those measuring points on which antibodies have been immobilized for which said cells express the corresponding CD antigens. The bound test cells are recorded optically in a space-resolved manner. The resulting pattern of measuring points occupied by test cells represents the immunophenotype of the patient from which the test cells are derived.

The antibody array comprises altogether 60 measuring points on an area of 0.72 cm×0.4 cm, to which in each case 5 nl of antibody solution were applied. A measuring point is approx. 400 μm in diameter. 100 μl of a cell suspension with a concentration of 107 test cells/ml were applied to the array. The authors report that at this concentration about 600 test cells per measuring point were bound, while at 106 test cells/ml approx. 100 test cells are bound.

In other words, only approx. 0.1 to 1% of the test cells present in the suspension are able to also bind to the measuring points. However, according to the knowledge of the inventors of the present application, such a low proportion of actually binding test cells has the problem of the results of the measurement being not reliable enough, for statistical reasons, in particular if it is intended to compare the results of the measurement for various measuring points not only qualitatively, as with typing of the immunophenotype in the publication mentioned, but also quantitatively, as with the functional assays mentioned at the outset. Said problem is even more serious if the number of available cells is small, i.e. if, for example only 105 cells/ml rather than 107 cells/ml are available, as is the case with many experimental approaches.

The fluorescence images of experimental results, depicted in said publication, are in addition conspicuous in that the measuring points vary greatly in size and partly are occupied very unevenly by test cells. According to the knowledge of the inventors of the present application, the process described in said publication, however, causes, due to these variations, a distortion of the results of the measurement so that the functional assays mentioned at the outset cannot be carried out with sufficient accuracy and reproducibility.

### SUMMARY OF THE INVENTION

Against this background, it is an object of the present invention to provide a process of the kind mentioned at the outset, in which the reliability and reproducibility between the results of the measurement of measuring points in one array and in various arrays is so high that it is possible to compare the results of the measurement in a reliable manner and to generate a reliable grading of the results of the measurement.

According to the invention, this object is achieved on the one hand by a process for carrying out functional assays on test cells comprising the following steps:
a) providing an array of measuring points which are separate from each other and to which in each case capture molecules to which said test cells can bind are immobilized,
b) generating a mixture of said test cells and of reference particles which are capable of binding to said capture molecules and which are distinguishable from said test cells,
c) contacting the mixture of step b) with the array so that test cells and reference particles can bind to each measuring point, and
d) determining the ratio of bound test cells of interest to bound reference particles for at least some of the measuring points.

This object underlying the invention is completely achieved in this manner.

The inventors of the present application have found that the reproducibility and reliability of the results of the measurement are a function of the varying size of the measuring points and of a lack of homogeneity of the immobilized capture molecules and that, by using the reference particles, it is possible to eliminate by calculation the influences of the different sizes of the measuring point areas and other inhomogeneities, for example of the local concentration of the capture molecules, from the results of the measurement. Thus, the inventors specifically do not follow the path which actually presents itself owing to the findings of the inventors, namely to minimize said variability by more complicated preparation processes which result in more uniform areas of the measuring points and in a more even local concentration of the capture molecules, but use reference particles.

Thus, in other words, the number of test cells bound per measuring point and thus the particular measured signal are, according to the finding of the inventors of the present application, in the known processes a function not only of the binding properties between the test cells and the capture molecules but also of the density of said capture molecules per measuring point, of the size of the measuring point area and of the homogeneity of the capture molecule concentration.

Nevertheless, due to the reference particles, the variability present in the size of the measuring point areas and the inhomogeneity of the applied capture molecules make it now possible to determine a gradation in the binding of the test cells to the various capture molecules with sufficient accuracy and reliability.

The quotient of the measured signal of the test cells and the measured signal of the reference particles at a particular measuring point may be taken as a measure for the binding of said test cells to the capture molecules of said measuring point, since the measured signal of the reference particles is, as it were, a measure of the number of capture molecules in one measuring point.

In this connection, “bound test cells of interest” means on the one hand test cells which have precipitated from the suspension on measuring points and adhere there. The ratio of bound test cells to bound reference particles is then used to enable the adhesion behaviors of the test cells to different capture molecules to be compared with one another.

On the other hand, it is also possible to investigate the rate of apoptosis or viability of test cells which, after adhesion to the capture molecules, are incubated with addition of particular substances or with treatment, for example by irradiation. After the incubation, the still vital test cells or those which are dying or have died are then recorded and normalized for each measuring point by means of the number of bound reference particles, making it possible to determine, for example, the influence of different capture molecules on the rate of apoptosis.

In this manner it is possible to study within the framework of functional assays, in addition to adhesion or rate of apoptosis, also other properties of the test cells or alterations in these properties. It is important here that the number of test cells “of interest” on a measuring point is normalized, as it were, by the number of the likewise bound reference cells and thus to be able to compare that first number with the number of test cells of interest on other measuring points. In adhesion studies, for example, the test cells of interest are any bound test cells, and in other functional assays the rate of apoptosis, the viability, the ability to bind to antibodies, to exchange signals, etc.

Reference particles which may be used here are artificial beads, for example latex beads, which carry surface molecules which enable binding to the capture molecules to be comparable to that of the surface molecules of the test cells. These beads can be prepared in an inexpensive and simple manner and may be stored for a long time; they are known from other applications in the prior art and have a size which may correspond to that of the test cells. The beads have an additional advantage in that their behavior of binding to the capture molecules is not influenced by substances added subsequently or, for example, by radioactive or UV irradiation so that, after mixing and, where appropriate, immobilizing test cells and beads, the influence of this measure on the test cells and, for example, on their binding or rate of apoptosis can be monitored, without influencing the binding of the beads, so that the reference is retained.

On the other hand, it is also possible to take as reference particles biological reference cells which can be distinguished by measurement, preferably optical measurement, from the test cells but which, like said test cells, bind to capture molecules. In this context, the reference cells may be untreated test cells which are distinguishable by measurement from the test cells to be investigated which have been treated prior to mixing.

However, it is also provided for both artificial beads and biological reference cells to be present in the reference particles. This enables a plurality of parameters to be corrected via, as it were, internal references.

The test cells and reference particles are preferably mixed with one another in a 1:1 ratio so as to hit the capture molecules with the same probability.

In one exemplary embodiment, the test cells can be distinguished by measurement, preferably optical measurement, from the reference particles in that said test cells are labeled with a different marker, preferably a fluorescent marker or a genetic marker, than said reference particles.

It is an advantage here that the array can be read out with two different excitation waves and/or emission filters, recording successively or simultaneously space-resolved optical signals from which the ratio of test cell of interest to reference particle can then be calculated.

It is also possible to label the test cells with a genetic marker such as, for example, a reporter gene such as GFP (green fluorescent protein). If the reference particles are reference cells, the latter may also be genetically labeled, either instead of the test cells or differently thereto.

In a further exemplary embodiment, the test cells can be distinguished by measurement from the reference particles by providing the test cells with a different radioactive marker than the reference particles.

It is also provided for both radioactive and optical labels to be used together, i.e. to label test cells and reference cells radioactively and optically, or mixed, i.e. to label, for example, the test cells optically and the reference particles radioactively.

The reference cells differ, preferably genetically, from the test cells in such a way that they do not react or react in a different known manner to substances and/or irradiations whose effect on said test cells is to be investigated.

Here, after mixing the test and reference cells, this mixture advantageously can be treated in the manner indicated, without also impairing binding to the capture molecules or other properties of the reference cells, which are investigated in the functional assays. In other words, while irradiation may result, for example, in a particular percentage of the test cells being killed or changing its binding properties to the capture molecules such that said binding to the capture molecules is worse or better, binding of the reference cells to the capture molecules remains unchanged and thus is a measure for the number of capture molecules per measuring point.

On the other hand, it is also possible to distribute a mixture of test cells and reference cells between two arrays and to irradiate only one array or contact it with a test substance, after or during incubation. The untreated array then serves as a reference for the action on test cells and on reference cells.

In this context, it is also possible to leave the reference cells untreated and only mix them with the test cells after treatment of the latter.

It is also possible to mix two or more types of reference particles with the test cells, with the different types of reference particles being distinguishable from one another and from the test cells, for example by way of three different “colors”. Thus, as a first type of reference particles, “beads” may be used which are not influenced by the treatment and which serve as a reference between different arrays and to use, as a second type of reference particles, reference cells which serve to eliminate by calculation the variation within an array.

Alternatively or additionally, each array may be provided with a reference measuring point to which only reference particles bind. This may be used as a reference between different arrays, said reference measuring point being isolated from the other measuring points so that only reference particles are applied to the former.

According to the invention, the object underlying the invention is achieved, on the other hand, by a process for carrying out functional assays on test cells comprising the following steps:
a) providing an array of measuring points which are separate from each other and to which in each case different capture molecules to which said test cells can bind are immobilized, with a measuring point with its assigned capture molecules is distributed in the array to a plurality of measuring areas which are arranged at various sites in said array,
b) contacting a suspension of test cells with the array so that test cells can bind to each measuring point,
c) recording in a space-resolved manner data representing the number of test cells of interest on the measuring areas,
d) calculating a measured value for at least one measuring point from the data of the measuring areas assigned to said measuring point.

The object underlying the invention is completely achieved in this manner too.

The inventors of the present application have found that the uneven deposition of test cells on the measuring points, as is apparent from the publication by Belov et al., loc cit, can be attributed not only to the inhomogeneity of the measuring points but also to the fact that the test cells are not homogeneously distributed in the suspension but are preferably deposited at particular sites on the array. However, this results in the local number of test cells on the array not being the same everywhere. Thus, in other words, despite strong binding between test cells and capture molecules, for example, a weaker measured signal may be produced for a particular measuring point than for a measuring point at which binding is weaker, because the local test cell concentration is lower at the first measuring point than at the second measuring point.

According to the finding of the inventors of the present application, distribution of a (logical) measuring point between a priority of measuring areas at different sites in the array, however, renders the statistical probability of adhesion for different measuring points, i.e. averaged over the assigned measuring areas, equally high.

If, in this context, the individual measuring areas become so small that, in a statistical sense, a sufficiently large number of test cells can no longer bind in order to generate a reliable measuring signal, then it is also possible here to use the reference particles and measures discussed in detail above, resulting in a synergistic effect.

In an ideal case, the areas produced for a measuring point are so large, based on the number of test cells in the suspension, that at least more than 50%, in an ideal case virtually 100%, of test cells from the supernatant can bind on the measuring points. To this end, according to the invention, a particular ratio between the area (F) of a measuring point and the number (N) of test cells in the suspension is required, which ratio is a function of the adhesion surface (H) of the particular cells and is chosen as follows:
F≧a×N×H, a=0.5, preferably approx. 1.0.

Adhesion surface here means the size of the area used by a cell to place itself on the substrate. For bacteria, this size is typically H=1 μm2 and for animal cells it is typically H>100 μm2. Thus, approx. 800 bacteria or 8 animal cells may be deposited on a measuring area of (F=800 μm2) so that in this case (F=800 μm2) the suspension applied to the array should contain at most N=800 bacteria or N=8 animal cells.

In order to achieve a higher measured signal, according to the invention, either a larger area F of a measuring point is chosen or a plurality of measuring areas are combined to give a logical measuring point having a total area F.

Under these conditions, virtually all test cells from the supernatant can bind, without impairing each other. This choice of ratio between the number and adhesion surface-determining type of test cells and the size of the area of a measuring point is also per se novel and inventive and results, together with either or both of the measures mentioned above, namely the reference particles and/or the distributed measuring areas, in a synergistic effect. When using reference particles, the above ratio is to be applied to the sum of the test cells present in the suspension and reference particles as follows:
F≧a×(NT×HT+NR×HR)
with NT and HT denoting the number and adhesion surface of the test cells and NR and HR denoting the number and adhesion surface of the reference particles, and a is a factor of 0.5, preferably approx. 1.0.

Thus, according to the finding of the inventors of the present application, for a given array with known measuring point areas, the amount of test cells and, where appropriate, reference particles in the suspension/mixture to be applied to said array must be chosen so as to maintain the above ratio in order to get to a situation in which virtually all test cells/reference particles can bind to a measuring point so that there is competition between the measuring points for the cells. This makes possible quantitative evaluations which would be distorted in the case of a larger number of test cells.

Said ratio is advantageous, for example, when few cells are available, for example in tumor biopsies, or when stem cell homing is to be investigated. A preference of test cells for particular capture molecules can be determined quantitatively only with the low numbers of test cells used according to the invention. This also applies if, in the case of mixed cell populations, a subpopulation is to be investigated separately.

In this context, the inventors of the present application showed that, with relatively large numbers of test cells, these also bind on substrates for which they have no specific preference.

Generally, preference is also given to agitating the array, after contacting with the suspension/mixture, i.e. during incubation with the test cells and, where appropriate, the reference particles, for example on a shaker or a rocker or by means of a pump, for example via microfluidic flow, in order to reduce local concentration differences in the test cells and, where appropriate, reference particles. Agitating furthermore continually delivers new test cells or reference particles to the measuring points so that a larger number thereof gets the opportunity of binding to capture molecules. In contrast to protein arrays, where the law of mass action applies, and said shaking would produce only limited advantages, shaking surprisingly increases considerably the number of bound test cells and, where appropriate, reference particles, as was shown by the inventors of the present application.

Against this background, this measure, in the case of a process mentioned at the outset, is also per se novel and inventive, but is preferably applied together with one or more of the measures mentioned above.

Overall, it is possible for the array to be applied either to a carrier plate, as is the case in WO 00/39580 and WO 02/02226, mentioned at the outset, or to be a logical array of individual beads which are loaded with capture molecules and which can be distinguished from one another in the usual manner, for example by color labels. A measuring point then corresponds either to one bead or to several beads which in each case represent a measuring area in the above sense.

If beads are used as an array, they are, in the simplest case, added to the solution/mixture and incubated with gentle agitation, for example on a shaker.

In particular applications, the test cells are subjected, before or after contacting with the array, to a treatment which is selected from the group consisting of: irradiating with high energy radiation, for example UV light or radioactive radiation, contacting with test substances such as, for example, pharmaceutical active agents, other cells, chemotherapeutics, components of extracellular matrix proteins, antibodies, lectins or other biopolymers.

Different capture molecules are immobilized on the measuring points which are preferably selected from the group consisting of: protein such as, for example, components of extracellular matrix proteins, receptors, ligands, polylysine, peptides of laminin sequences, control peptides, peptidomimetics, antibodies, lectins, antigens, and allergens.

A further object of the invention is a kit having an array of measuring points which are separate from one another and to which capture molecules to which test cells can bind are immobilized and comprising reference particles which bind to said capture molecules.

In this context, the reference particles are preferably the reference particles described in more detail above, with further preference being given to different capture molecules being immobilized on the measuring points which are preferably selected from the group consisting of: protein such as, for example, components of extracellular matrix proteins, receptors, ligands, polylysine, peptides of laminin sequences, control peptides, peptidomimetics, antibodies, lectins, antigens, allergens, nucleic acids and nucleotides.

In this context, either the array is applied to a carrier plate or it is a logical array of individual beads loaded with capture molecules, with further preference being given to at least one measuring point with its assigned capture molecules being distributed between a plurality of measuring areas in the array, which are arranged at various sites in said array.

Further advantages and features ensue from the following description and the attached figures.

It will be appreciated that the aforementioned features and the features still to be illustrated below can be used not only in the combination indicated in each case but also in other combinations or on their own, without leaving the scope of the present invention.

Exemplary embodiments of the invention are depicted in the figures and will be illustrated in more detail in the following description.

### BRIEF DESCRIPTION OF THE DRAWINGS

FIG. 1 depicts a diagrammatic example of an array of measuring points arranged on a carrier plate, said measuring points being distributed between various measuring areas;

FIG. 2 depicts a diagrammatic side view of the carrier plate of FIG. 1;

FIG. 3 depicts images of test cells and reference cells bound to a measuring point of the array of FIG. 1, with A) being the bright field image of test cells and reference cells, B) being a fluorescence image of the test cells and the reference cells, C) a fluorescence image of the test cells and D) a fluorescence image of the reference cells;

FIG. 4 depicts various fluorescence images depicting the binding of test cells to various measuring points, which were agitated (B, D and F) and not agitated (A, C and E) during incubation;

FIG. 5 depicts a diagram showing binding of test cells to various capture molecules as a function of the number of cells used in the suspension applied; and

FIG. 6 depicts a diagram showing colonization of a measuring area as a function of the number of cells used in the suspension applied, with and without agitation.

### DETAILED DESCRIPTION

### EXAMPLE 1

### Carrier Plate with an Array of Measuring Points

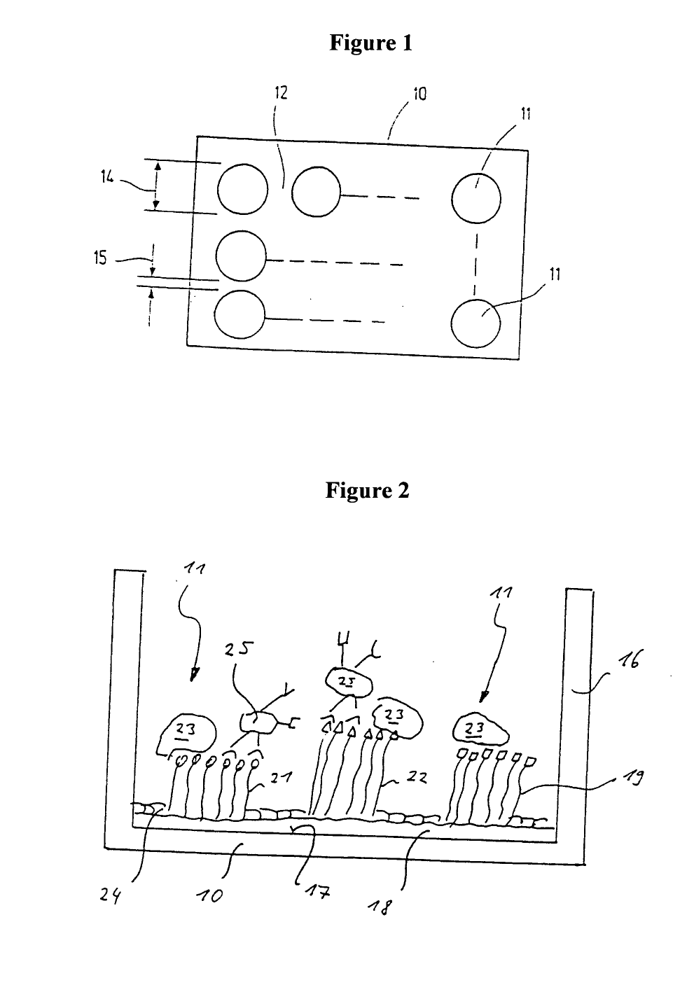

In FIG. 1, “10” indicates a rectangular carrier plate made from glass or plastic, on which some measuring areas 11 are arranged here by way of example in an array, to which areas biological cells—test cells hereinbelow—or reference particles, not shown in FIG. 1, can bind. Regions 12 of the carrier plate 10, to which test cells and reference particles cannot bind, are arranged between the measuring areas 11.

The measuring areas are 500 μm in diameter and the distance between their edges is 250 μm, resulting in their centers being 750 μm apart. In this way, 96 measuring areas 11 can be fitted on a carrier plate 10 having an edge length of 6 mm×9 mm.

As the diagrammatic side view of FIG. 2 shows, the carrier plate can be designed as the bottom plate of a cell culture vessel. On its section 17, the carrier plate 10 carries a functionalized surface 18 on which capture molecules 19, 21, 22 are immobilized in the measuring points 11.

The capture molecules 19, 21, 22 usually differ from measuring area 11 to measuring area 11, it being possible for various measuring areas 11 to be combined to give a logical measuring point. The measuring area 11 of a logical measuring point carry identical capture molecules 19, 21 or 22 and are randomly distributed across the carrier plate 10.

It is possible for test cells 23, whose behavior of binding to the capture molecules 19, 21, 22 or whose reaction to costimulation by capture molecules 19 and test substances and, respectively, to a treatment such as, for example, irradiation, is to be investigated, to bind to the capture molecules 19, 21 and 22.

In the regions 12 between the measuring areas 11, the functionalized surface 18 is blocked by molecules 24 so as to enable the test cells 23 to be bound only in the region of the measuring areas 11.

In FIG. 2, a reference particle which binds to the capture molecules 22 is indicated at 25. Suitable reference particles 25 are biological cells or else plastic beads which carry on their surface molecules with which they bind to the capture molecules 19, 21, 22. The reference particles 25 are intended as an internal reference in order to be able to eliminate by calculation variations in the size of the measuring area 11 or the number of capture molecules in the measuring area, both within one array and between various arrays.

Example 1 of WO 02/02226 mentioned above, whose disclosure is hereby explicitly referred to, describes how such a carrier plate 10 with measuring area 11 can be prepared.

### EXAMPLE 2

### Incubating a Mixture of Test Cells and Reference Cells on an Array with Measuring Points Distributed Between Various Measuring Areas

For this experiment, test and reference cells are labeled with various membrane dyes.

Test cells used here are hu AO SMC and GLZ which are labeled using the Vibrant Dil Red Fluorescent Cell Linker Kit (V22885Y MoBiTec) according to the manufacturer's protocols.

The reference cells used are PC12 which are labeled using the Vibrant DiO Green Fluorescent Cell Linker Kit (V22886Y MoBiTec) according to the manufacturer's protocols.

To depict test and reference cells in an image, both cell types were stained blue with Vibrant Cell Labeling Solution DAPI.

The capture molecules immobilized in the measuring areas were various matrix proteins.

The test and reference cells were mixed in a 1:1 ratio, the mixture was applied to the carrier plate containing the array and the carrier plate was then incubated in an incubator at 37° C. for 4 h.

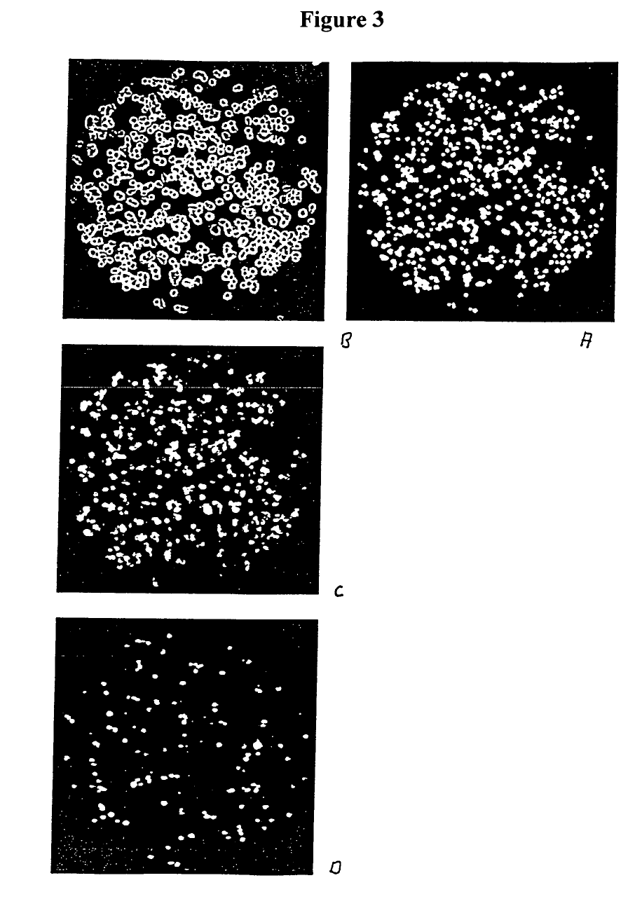

FIG. 3 shows, for a measuring point with laminin of human placenta as capture molecule by way of example, that both test cells (GLZ) and reference cells (PC 12) bind to said measuring point but that the number of reference cells is distinctly smaller than the number of test cells. The measuring point is shown to be uniformly occupied with capture molecules.

FIG. 3A depicts a bright field image for both cell types, FIG. 3B depicts a fluorescence image of the measuring point, using a blue filter for test and reference cells after DAPI staining, FIG. 3C depicts a fluorescence image using a red filter which is transparent for emission from the test cells, and FIG. 3D depicts a corresponding image using a green filter which is transparent for emission from the reference cells.

The table below lists, for some measuring points, in each case with laminin (human placenta) as capture molecule for a mixed suspension of test and reference cells by way of example, the total number and percentage of the cells bound in each case, with, for PC 12 with GLZ, in each case 100 000 cells of each type being applied to one array and, for PC 12 with hu AO SMC, in each case 35 000 cells of each type being applied to one array.

| Measuring | | Measuring | | Measuring | | Measuring | |
|---|---|---|---|---|---|---|---|
| point 1 | | point 2 | | point 3 | | point 4 | |
| | Relative | | Relative | | Relative | | Relative |
| | proportion | | proportion | | proportion | | proportion |
| Number | [%] | Number | [%] | Number | [%] | Number | [%] |

Colonization of laminin measuring areas by test cells (glioma cell
line GLZ) and reference cells (neuronal cell line PC12)

| | | | | | | | | |
|---|---|---|---|---|---|---|---|---|
| Total | 53 | 100 | 219 | 100 | 446 | 100 | 569 | 100 |
| GLZ | 34 | 64 | 153 | 70 | 334 | 75 | 427 | 75 |
| PC12 | 19 | 36 | 66 | 30 | 112 | 25 | 144 | 25 |

Colonization of laminin measuring areas by test cells (smooth muscle
cells, huAO SMC) and reference cells (neuronal cell line PC12)

| | | | | | | | | |
|---|---|---|---|---|---|---|---|---|
| Total | 404 | 100 | 658 | 100 | 666 | 100 | 655 | 100 |
| huAO | 70 | 17 | 78 | 12 | 61 | 9 | 77 | 12 |
| SMC | | | | | | | | |
| PC12 | 335 | 83 | 579 | 88 | 606 | 91 | 578 | 88 |

Table: Adhesion of test and reference cells; the number of cells bound per measuring point and the particular relative proportion of the colonized area are indicated.

Although there are extreme deviations in the number of bound cells between individual measuring points, the percentage of bound test cells is approximately constant within the degree of variation common for biological measurements. While, for example, only 53 cells have bound to measuring point 1 and even 569 cells in total have bound to measuring point 4, in each case 64% and 75%, respectively, of the bound cells were test cells (GLZ). For measuring points 2 and 3, the ratio, with 70% and 75%, respectively, was also in this range.

Although this constant ratio is changed in that binding of PC 12 to the capture molecules is poorer in comparison with GLZ than in comparison with hu AO SMC, it remains, however, even here sufficiently constant, with from 9% to 17%.

In other words, the ratio between the two particular cell types is approximately constant when the ratio F≧N×H is maintained, independently of the number of cells per measuring area and independently of whether the two cell types compete for a capture molecule. Thus it is possible to eliminate by calculation the variation between measuring points by using reference cells.

The percentage or else the ratio between bound test and reference cells is thus a more reliable measure for binding of the test cells to the individual measuring points than the absolute number of bound test cells.

### EXAMPLE 3

### Incubation with and without Shaking

Test cells (PC 12) were incubated at a concentration of from 0.5 to 50×105 cells/ml on measuring areas of an area of 280 000 μm2 with various capture molecules, namely collagen I, collagen II and collagen III, for in each case 4 h, with, in one case, the carrier plates being shaken during incubation and, in the other case, being left resting. For shaking, the carrier plate was manually agitated at 10 min intervals, in order to mix the cell suspension on top of the arrays.

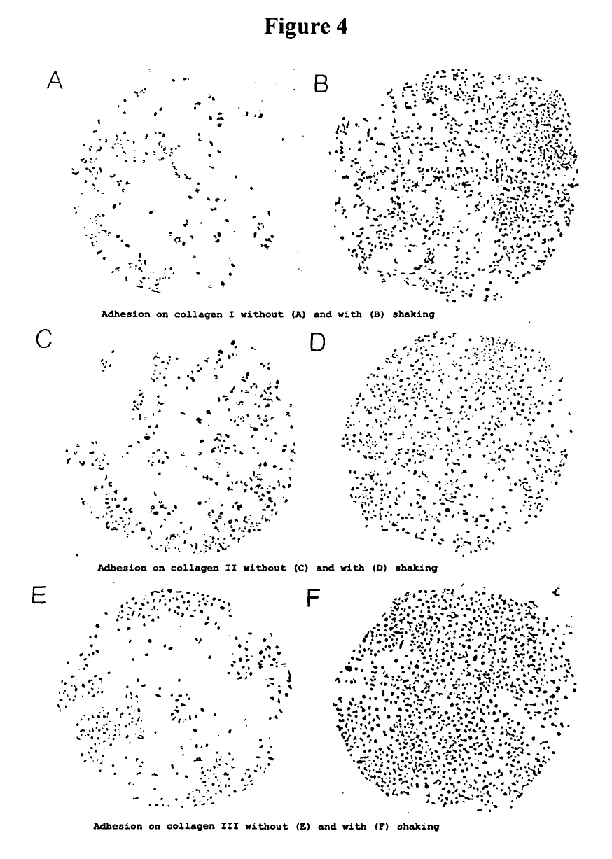

FIG. 4 depicts in the top row binding to collagen I, in the middle row binding to collagen II and in the bottom row binding to collagen III. On the left hand side, incubation without shaking is shown in each case at A, C and E and on the right hand side incubation with shaking is shown at B, D and E.

The images reveal that shaking results in a markedly increased and also markedly more uniform binding of the test cells to the capture molecules.

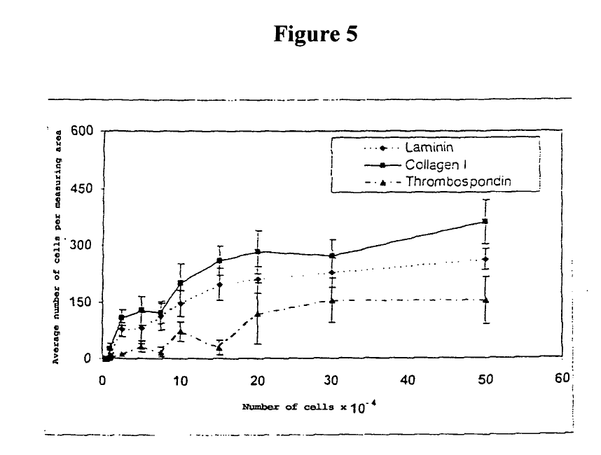

FIG. 5 depicts the number of cells per measuring area as a function of the number of cells used and binding of the test cells to the three capture molecules mentioned in FIG. 5.

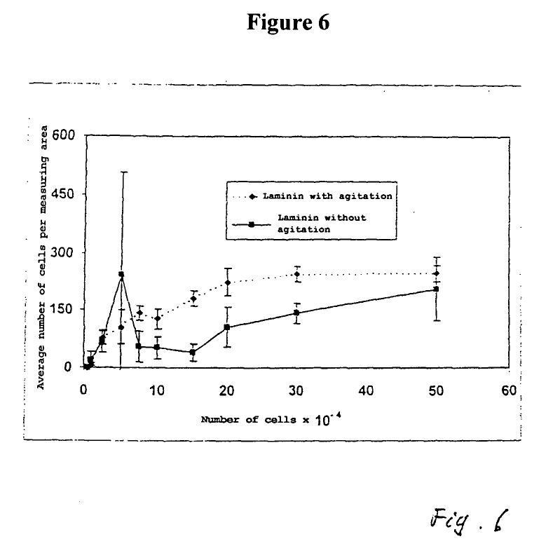

FIG. 6 depicts the number of cells per measuring area as a function of the number of cells used and binding to the substrate laminin and the influence of substrate movement.

It is revealed that shaking provides maximum colonization of a measuring area by cells. “Saturation” of the measuring areas occurs already at a concentration of 20×105 cells/ml, which is obvious from the fact that, from this concentration onward, the number of bound cells basically no longer increases.

Furthermore, the left branches of the curves reveal that at low concentrations, i.e. at a lower number of cells per measuring point, basically no binding to thrombospondin occurs, but that virtually all cells bind to laminin or collagen I. This is also to be expected in this way, since PC12 binds considerably more weakly to thrombospondin than to the other capture molecules. Only a large number of cells in the supernatant attenuates the effect of competition and the cells also bind to thrombospondin.

### EXAMPLE 4

### Determination of the Sensitivity of Cells in their Natural Microenvironment

An example of how the process of the invention can be used is the investigation of the manner in which tumor and normal tissues react to irradiation or the addition of toxic substances as a function of their natural microenvironment. Cells whose sensitivity to radiation is assayed in a cell culture without addition of extracellular matrix components (ECM) are known to be more sensitive to radiation than parallel cultures which have been cultured on ECM, meaning that the composition of the extracellular matrix is crucially important for the cell-specific reactivity both of tumor and of normal tissues. It is moreover possible to optimize further the combined action of radiotherapy and chemotherapy in the natural microenvironment.

These experiments were carried out by using the ECM as capture molecules. The test cells are applied in mixed suspension with reference cells to an array of various capture molecules and the number of bound test cells and of bound reference cells is determined and the normalized number of test cells per measuring point is calculated therefrom. The test cells are then treated with staurospondin and incubated. After a certain incubation time, the number of dead test cells per measuring point is determined and the rate of apoptosis is determined from this number and from the normalized number determined prior to incubation.

Experiments with staurosporin-induced apoptosis in PC12 cells showed that these cells are markedly better protected from the harmful actions of staurosporin by collagen IV and laminin, i.e. their natural substrates, than by PLL (poly-L-lysine), which probably has no protective action. The rate of apoptosis of test cells on lamin increased by a factor of 4 after staurospondin treatment, while test cells growing on PLL had an increase by a factor of 15. This difference could be determined only by using the reference cells.

1. A process for carrying out functional assays on test cells comprising the following steps:
a) providing an array of measuring points which are separate from each other and to which in each case capture molecules to which said test cells can bind are immobilized,
b) generating a mixture of said test cells and of reference particles which are capable of binding to said capture molecules and which are distinguishable from said test cells,
c) contacting the mixture of step b) with the array so that test cells and reference particles can bind to each measuring point, and
d) determining the ratio of bound test cells of interest to bound reference particles for at least some of the measuring points.

2. The process as claimed in claim 1, wherein in step b) test cells and reference particles are mixed in a 1:1 ratio.

3. The process as claimed in claim 1, wherein the reference particles comprise artificial beads, for example latex beads, which carry surface molecules which enable binding to the capture molecules to be comparable to that of the surface molecules of the test cells.

4. The process as claimed in claim 1, wherein the reference particles comprise biological reference cells which are distinguishable by measurement, preferably optical measurement, from the test cells but which, like said test cells, bind to capture molecules.

5. The process as claimed in claim 4, wherein the reference cells differ from the test cells in such a way that they do not react or react in a different known manner to substances and/or irradiations whose effect on said test cells is to be investigated.

6. The process as claimed in claim 1, wherein the test cells are optically distinguishable from the reference particles in that said test cells are labeled with a different marker of said reference particles.

7. The process as claimed in claim 6, wherein the marker is selected from the group consisting of: a fluorescent marker and a genetic marker.

8. The process as claimed in 1, wherein the test cells are distinguishable by measurement from the reference particles in that said test cells are provided with a different radioactive marker than said reference particles.

9. The process as claimed in claim 1, wherein at least one measuring point with its assigned capture molecules is distributed in the array to a plurality of measuring areas which are arranged at various sites in said array.

10. The process as claimed in claim 9, wherein the at least one measuring point, in each case one measured value is calculated for the test cells and the reference particles from measured values which are determined for the assigned measuring areas.

11. The process as claimed in claim 1, wherein a ratio exists between the area F of a measuring point and the number NT of test cells and the number NR of reference particles in the mixture, which ratio is a function of the adhesion surface HT of the test cells and the adhesion surface HR of the reference particles and which is chosen as follows:
F≧a×(NT×HT+NR×HR),a=0.5.

12. The process as claimed in claim 11, wherein factor a is approx. 1.0.

13. A process for carrying out functional assays on test cells comprising the following steps:
a) providing an array of measuring points which are separate from each other and to which in each case different capture molecules to which said test cells can bind are immobilized, with at least one measuring point with its assigned capture molecules is distributed in the array to a plurality of measuring areas which are arranged at various sites in said array,
b) contacting a suspension of test cells with the array so that test cells can bind to each measuring point,
c) recording in a space-resolved manner data representing the number of test cells of interest on the measuring areas,
d) calculating a measured value for at least one measuring point from the data of the measuring areas assigned to said measuring point.

14. The process as claimed in claim 13, wherein a ratio exists between F, the sum of the measuring areas of a measuring point, and N, the number of test cells in the suspension, which ratio is a function of the adhesion surface H of said test cells and which is chosen as follows:
F≧a×N×H,a=0.5.

15. The process as claimed in claim 14, wherein factor a is approx. 1.0.

16. A process for carrying out functional assays on test cells comprising the following steps:
a) providing an array of measuring points which are separate from each other and to which in each case capture molecules to which said test cells can bind are immobilized,
b) contacting a suspension of test cells with the array so that test cells can bind to each measuring point, with there existing a ratio between the area F of a measuring point and the number N of test cells in the suspension, which ratio is a function of the adhesion surface H of said test cells and which is chosen as follows:
F>a×N×H,a=0.5.

17. The process as claimed in claim 16, wherein factor a is approx. 1.0.

18. The process as claimed in claim 1, wherein the array, after having been contacted with the test cells and, where appropriate, the reference particles, is agitated on a shaker or a rocker.

19. A process for carrying out functional assays on test cells comprising the following steps:
a) providing an array of measuring points which are separate from each other and to which in each case capture molecules to which said test cells can bind are immobilized,
b) contacting a suspension of test cells with the array so that test cells can bind to each measuring point, and
c) incubating the array with the suspension, agitating said array on a shaker or on a rocker.

20. The process as claimed in claim 1, wherein either the array is applied to a carrier plate or it is a logical array of individual beads loaded with capture molecules.

21. The process as claimed in claim 1, wherein the test cells, before or after contacting with the array, are subjected to a treatment selected from the group consisting of: irradiating with high energy radiation, for example UV light or radioactive radiation, contacting with test substances such as, for example, pharmaceutical active agents, other cells, chemotherapeutics, components of extracellular matrix proteins, antibodies, lectins or other biopolymers.

22. The process as claimed in claim 1, wherein different capture molecules are immobilized on the measuring points which are selected from the group consisting of: protein such as, for example, components of extracellular matrix proteins, receptors, ligands, polylysine, peptides of laminin sequences, control peptides, peptidomimetics, antibodies, lectins, antigens, and allergens.

23. A kit comprising an array of measuring points which are separate from one another and to which capture molecules to which test cells can bind are immobilized and comprising reference particles which bind to said capture molecules.

24. The kit as claimed in claim 23, wherein the reference particles are artificial beads and/or biological reference cells.

25. The kit as claimed in claim 23, wherein different capture molecules are immobilized on the measuring points which are selected from the group consisting of: protein such as, for example, components of extracellular matrix proteins, receptors, ligands, polylysine, peptides of laminin sequences, control peptides, peptidomimetics, antibodies, lectins, antigens, and allergens.

26. The kit as claimed in claim 23, wherein either the array is applied to a carrier plate or it is a logical array of individual beads loaded with capture molecules.

27. The kit as claimed in claim 23, wherein at least one measuring point with its assigned capture molecules is distributed in the array to a plurality of measuring areas which are arranged at various sites in said array.
